SEQUENCE LISTING (15 sequences)

SEQ ID NO: 1 - protein, 450 aa, synthetic construct
QVQLQESGPGLVKPSETLSLTCTVSEGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 448 aa, synthetic construct
QVQLVQSGSELKKPGASVKVSCKASGYTFTTNALNWVRQAPGQGLEWMGWINTHTGNPTYAQGFIGRFVFSLDTSVSTAYLQIRSLKAEDTAVYYCAREPNWGVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICKVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 121 aa, synthetic construct
QVQLQESGPGLVKPSETLSLTCTVSEGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSS

SEQ ID NO: 5 - protein, 119 aa, synthetic construct
QVQLVQSGSELKKPGASVKVSCKASGYTFTTNALNWVRQAPGQGLEWMGWINTHTGNPTYAQGFIGRFVFSLDTSVSTAYLQIRSLKAEDTAVYYCAREPNWGVYFDYWGQGTLVTVSS

SEQ ID NO: 6 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 7 - protein, 5 aa, synthetic construct
YHFWS

SEQ ID NO: 8 - protein, 16 aa, synthetic construct
YIVYSGSYNVNPSLKT

SEQ ID NO: 9 - protein, 13 aa, synthetic construct
GGYTGYGGDWFDP

SEQ ID NO: 10 - protein, 5 aa, synthetic construct
TNALN

SEQ ID NO: 11 - protein, 17 aa, synthetic construct
WINTHTGNPTYAQGFIG

SEQ ID NO: 12 - protein, 10 aa, synthetic construct
EPNWGVYFDY

SEQ ID NO: 13 - protein, 11 aa, synthetic construct
RASQSISSYLN

SEQ ID NO: 14 - protein, 7 aa, synthetic construct
AASSLQS

SEQ ID NO: 15 - protein, 9 aa, synthetic construct
QQSYSTPPT

(19) United States
(12) Patent Application Publication
(10) Pub. No.: US 2024/0101718 A1
(43) Pub. Date: Mar. 28, 2024

(54) ANTI-PD-1/LAG-3 BISPECIFIC ANTIBODIES AND USES THEREOF

(71) Applicant: Incyte Corporation, Wilmington, DE (US)

(72) Inventors: Richard L. Schaub, West Chester, PA (US); Patrick Mayes, Wilmington, DE (US)

(21) Appl. No.: 18/373,648

(22) Filed: Sep. 27, 2023

Related U.S. Application Data
(60) Provisional application No. 63/410,709, filed on Sep. 28, 2022.

(51) Int. Cl. C07K 16/46; A61P 35/04

(52) U.S. Cl. CPC C07K 16/468; A61P 35/04; A61K 2039/545; C07K 2317/565

(57) ABSTRACT

The present disclosure describes a bispecific antibody targeting PD-1 and LAG-3 for use in the treatment of advanced malignancies such as melanoma and squamous cell carcinoma of the head and neck.

### CROSS-REFERENCE TO RELATED APPLICATIONS

This application claims the benefit of priority to U.S. Application No. 63/410,709, filed on Sep. 28, 2022, the contents of which are hereby incorporated by reference.

### SEQUENCE LISTING

This application contains a Sequence Listing that has been submitted electronically as an XML file named “20443-0786001_SL_ST26. XML.” The XML file, created on Sep. 27, 2023, is 15,417 bytes in size. The material in the XML file is hereby incorporated by reference in its entirety.

### BACKGROUND

Prior to approval of the anti-CTLA-4 antibody ipilimumab in 2011, effective therapies for the treatment of advanced or metastatic melanoma were limited and included chemotherapy and immunotherapy with high-dose IL-2. Following the approval of ipilimumab, two additional classes of drugs have entered the treatment paradigm: agents targeting tyrosine kinase pathways, including BRAF and MEK (e.g., vemurafenib, dabrafenib, trametinib, and cobimetinib), and monoclonal antibodies that block the PD-1 receptor (e.g., nivolumab and pembrolizumab). Although many of these therapies have demonstrated overall survival benefit both independently and in combination, there are still areas of high unmet medical need for this population (Hodi et al., N Engl J Med 2010;363:711-23, McArthur et al., Lancet Oncol 2014;15:323-32, Robert et al., N Engl J Med 2015;372:320-30, Wolchok et al., J Clin Oncol 2013;31 (suppl): Abstract 0790). Many patients experience a relapse shortly after initiating treatment or are not eligible for treatment due to tumor mutation status requirements (e.g., BRAF V600 mutation is present in approximately 50% of melanoma tumors: Chapman et al., N Engl J Med 2011;364:2507-16, Hauschild et al., Lancet 2012;380:358-65). Therefore, new therapeutic approaches are needed for treatment of melanoma.

Most patients with squamous cell carcinoma of the head and neck (SCCHN) are diagnosed with locally advanced disease, and more than half develop locoregional or distant relapse despite multimodal treatment approaches (e.g., surgical resection, radiotherapy, and adjuvant chemotherapy as radiosensitizer: Argiris et al., Lancet 2008;371:1695-1709, Ortiz-Cuaran et al., Front Oncol 2021;11:614332). For patients with recurrent or metastatic SCCHN, platinum-based chemotherapy and/or pembrolizumab are often used as first-line therapy. When pembrolizumab was used as first-line therapy for recurrent or metastatic SCCHN, the response rate was 19% in patients with PD-L1-positive tumors (CPS≥1;16.9% in the total population) for pembrolizumab monotherapy and 36.4% when combined with platinum-based chemotherapy (Rischin et al., J Clin Oncol 2019;37(suppl): Abstract6000). An estimated 85% to 95% of patients with recurrent or metastatic SCCHN have no response to these treatments or have a response that is followed by disease progression (Chow, N Engl J Med 2020;382:60-72).

New therapeutic approaches are needed for treatment of melanoma, SCCHN, and other advanced malignancies.

### SUMMARY

The disclosure features a method of treating a disorder in a human subject in need thereof, wherein the disorder is selected from the group consisting of a melanoma, non-small cell lung cancer (NSCLC), small cell lung cancer, malignant pleural mesothelioma, hormone receptor-positive/human epidermal growth factor receptor 2 (HER2) negative breast cancer, triple-negative breast cancer, nasopharyngeal carcinoma, esophageal carcinoma, hepatocellular carcinoma, renal cell carcinoma, urothelial carcinoma, a B-cell lymphoma, a high microsatellite instability (MSI-H)/deficient mismatch repair (dMMR) solid tumor, a squamous cell carcinoma of the head and neck (SCCHN), anal carcinoma, cervical cancer, a DNA polymerase epsilon mutated solid tumor, and clear cell ovarian or endometrial carcinoma. The method entails administering to the human subject a therapeutically effective amount of a bispecific antibody that binds to human PD-1 and human LAG-3, wherein the bispecific antibody comprises:
an anti-human PD-1 binding domain comprising a PD-1 heavy chain variable region and a PD-1 light chain variable region, wherein the PD-1 heavy chain variable region comprises heavy chain CDRI (HCDR1) comprising the amino acid sequence YHFWS (SEQ ID NO:7), heavy chain CDR2 (HCDR2) comprising the amino acid sequence YIVYSGSYNVNPSLKT (SEQ ID NO:8), and heavy chain CDR3 (HCDR3) comprising the amino acid sequence GGYTGYGGDWFDP (SEQ ID NO:9), and wherein the PD-1 light chain variable region comprises light chain CDRI (LCDR1) comprising the amino acid sequence RASQSISSYLN (SEQ ID NO: 13), light chain CDR2 (LCDR2) comprising the amino acid sequence AASSLQS (SEQ ID NO: 14), and light chain CDR3 (LCDR3) comprising the amino acid sequence QQSYSTPPT (SEQ ID NO:15); and
an anti-human LAG-3 binding domain comprising a LAG-3 heavy chain variable region and a LAG-3 light chain variable region, wherein the LAG-3 heavy chain variable region comprises HCDR1 comprising the amino acid sequence TNALN (SEQ ID NO:10), HCDR2 comprising the amino acid sequence WINTHTGNPTYAQGFIG (SEQ ID NO:11), and HCDR3 comprising the amino acid sequence EPNWGVYFDY (SEQ ID NO:12), and wherein the LAG-3 light chain variable region comprises LCDRI comprising the amino acid sequence RASQSISSYLN (SEQ ID NO:13), LCDR2 comprising the amino acid sequence AASSLQS (SEQ ID NO: 14), and LCDR3 comprising the amino acid sequence QQSYSTPPT (SEQ ID NO:15).

In some embodiments, the PD-1 heavy chain variable region comprises the amino acid sequence

(SEQ ID NO: 4)
QVQLQESGPGLVKPSETLSLTCTVSEGSIGYHFWSWIRQPPGRGLEWIG
YIVYSGSYNVNPSLKTRVTMSVDTSKNQFSLNLRSVTAADTAVYYCARG
GYTGYGGDWFDPWGQGTLVTVSS

and the LAG-3 heavy chain variable region comprises the amino acid sequence

(SEQ ID NO: 5)
QVQLVQSGSELKKPGASVKVSCKASGYTFTTNALNWVRQAPGQGLEWMG
WINTHTGNPTYAQGFIGRFVFSLDTSVSTAYLQIRSLKAEDTAVYYCAR
EPNWGVYFDYWGQGTLVTVSS

In some embodiments, the PD-1 light chain variable region and the LAG-3 light chain variable region each comprise the amino acid sequence

(SEQ ID NO: 6)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK

In some embodiments, the PD-1 heavy chain variable region comprises the amino acid sequence

(SEQ ID NO: 4)
QVQLQESGPGLVKPSETLSLTCTVSEGSIGYHFWSWIRQPPGRGLEWIG
YIVYSGSYNVNPSLKTRVTMSVDTSKNQFSLNLRSVTAADTAVYYCARG
GYTGYGGDWFDPWGQGTLVTVSS

the PD-1 light chain variable region comprises the amino acid sequence

(SEQ ID NO: 6)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK

the LAG-3 heavy chain variable region comprises the amino acid sequence

(SEQ ID NO: 5)
QVQLVQSGSELKKPGASVKVSCKASGYTFTTNALNWVRQAPGQGLEWMG
WINTHTGNPTYAQGFIGRFVFSLDTSVSTAYLQIRSLKAEDTAVYYCAR
EPNWGVYFDYWGQGTLVTVSS

and the LAG-3 light chain variable region comprises the amino acid sequence

(SEQ ID NO: 6)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK

In some embodiments, the bispecific antibody comprises a PD-1 heavy chain, a PD-1 light chain, a LAG-3 heavy chain, and a LAG-3 light chain, wherein the PD-1 heavy chain comprises the amino acid sequence set forth in SEQ ID NO: 1, the PD-1 light chain light chain comprises the amino acid sequence set forth in SEQ ID NO:3, the LAG-3 heavy chain comprises the amino acid sequence set forth in SEQ ID NO:2, and the LAG-3 light chain light chain comprises the amino acid sequence set forth in SEQ ID NO:3.

In some embodiments, the human subject has a melanoma (e.g., unresectable or metastatic melanoma).

In some embodiments, the melanoma has a V600-activating BRAF mutation.

In some embodiments, the human subject has a SCCHN (e.g., recurrent or metastatic SCCHN). In some embodiments, the SCCHN is PD-L1+ SCCHN. In some embodiments, the SCCHN is a primary squamous tumor of the oral cavity, oropharynx, hypopharynx, or larynx.

In some embodiments, the human subject has NSCLC.

In some embodiments, the human subject has small cell lung cancer.

In some embodiments, the human subject has malignant pleural mesothelioma.

In some embodiments, the human subject has hormone receptor-positive/HER2 negative breast cancer.

In some embodiments, the human subject has triple-negative breast cancer.

In some embodiments, the human subject has nasopharyngeal carcinoma.

In some embodiments, the human subject has esophageal carcinoma.

In some embodiments, the human subject has hepatocellular carcinoma.

In some embodiments, the human subject has renal cell carcinoma.

In some embodiments, the human subject has urothelial carcinoma.

In some embodiments, the human subject has a B-cell lymphoma. In some embodiments, the B-cell lymphoma is diffuse large B-cell lymphoma (DLBCL) or primary mediastinal B-cell lymphoma (PMBCL).

In some embodiments, the human subject has a MSI-H/dMMR solid tumor.

In some embodiments, the human subject has a SCCHN, anal carcinoma, or cervical cancer (e.g., human papilloma virus (HPV)-positive SCCHN, anal carcinoma, or cervical cancer).

In some embodiments, the human subject has a DNA polymerase epsilon mutated solid tumor. In some embodiments, the DNA polymerase epsilon mutated solid tumor comprises DNA polymerase epsilon mutations P286R and/or V411L.

In some embodiments, the human subject has clear cell ovarian or endometrial carcinoma.

In some embodiments, the human subject has experienced disease progression after prior treatment. In some embodiments, the prior treatment comprises anti-PD-(L)1 therapy and/or and platinum-based therapy.

In some embodiments, the disorder is nonamenable to curative treatments or procedures.

In some embodiments, the bispecific antibody is administered intravenously.

In some embodiments, the bispecific antibody is administered at a dose of about 20 mg, about 50 mg, about 150 mg, about 450 mg, about 900 mg, about 1800 mg, or about 2500 mg.

In some embodiments, the bispecific antibody is administered once every three weeks.

In some embodiments, the bispecific antibody is administered intravenously at a dose of about 20 mg, about 50 mg, about 150 mg, about 450 mg, about 900 mg, about 1800 mg, or about 2500 mg.

In some embodiments, the bispecific antibody is administered intravenously once every three weeks at a dose of about 20 mg, about 50 mg, about 150 mg, about 450 mg, about 900 mg, about 1800 mg, or about 2500 mg.

In some embodiments, the bispecific antibody comprises a PD-1 heavy chain, a PD-1 light chain, a LAG-3 heavy chain, and a LAG-3 light chain, wherein the PD-1 heavy chain comprises the amino acid sequence set forth in SEQ ID NO:1, the PD-1 light chain light chain comprises the amino acid sequence set forth in SEQ ID NO:3, the LAG-3 heavy chain comprises the amino acid sequence set forth in SEQ ID NO:2, and the LAG-3 light chain light chain comprises the amino acid sequence set forth in SEQ ID NO:3, and wherein the bispecific antibody is administered intravenously at a dose of about 20 mg, about 50 mg, about 150 mg, about 450 mg, about 900 mg, about 1800 mg, or about 2500 mg.

In some embodiments, the bispecific antibody comprises a PD-1 heavy chain, a PD-1 light chain, a LAG-3 heavy chain, and a LAG-3 light chain, wherein the PD-1 heavy chain comprises the amino acid sequence set forth in SEQ ID NO:1, the PD-1 light chain light chain comprises the amino acid sequence set forth in SEQ ID NO:3, the LAG-3 heavy chain comprises the amino acid sequence set forth in SEQ ID NO:2, and the LAG-3 light chain light chain comprises the amino acid sequence set forth in SEQ ID NO:3, and wherein the bispecific antibody is administered intravenously once every three weeks at a dose of about 20 mg, about 50 mg, about 150 mg, about 450 mg, about 900 mg, about 1800 mg, or about 2500 mg.

Other features and advantages of the invention will be apparent from the following detailed description, and from the claims.

### DETAILED DESCRIPTION

A bispecific antibody targeting PD-1 and LAG-3 can be used to treat advanced malignancies such as melanoma and squamous cell carcinoma of the head and neck.

### Anti-PD-1/LAG-3 Bispecific Antibodies

Programmed Cell Death 1 protein (PD-1, also known as CD279) is a cell surface receptor expressed on CD4+ and CD8+ T cells, B cells, NK cells, and myeloid-derived cells. PD-1 binds to two distinct ligands, PD-L1 and PD-L2, which differ in their expression patterns. PD-L1 (also known as B7-H1 or CD274) is expressed on hematopoietic cells such as T cells, B cells, dendritic cells, and macrophages, as well as an array of peripheral tissues, and PD-L1 expression levels are inducible by interferons. In contrast, PD-L2 (also known as B7-DC or CD273) expression is generally restricted to professional APCs and inducible by IL-4 and IL-10, depending on the lineage subset of the APC.

Binding of PD-1 to either PD-L1 or PD-L2 on T cells or B cells results in clustering with TCRs or BCRs and transient association with SH2 domain-containing tyrosine phosphatase 2. In turn, this induces a negative signal by dephosphorylating effector molecules that drive positive TCR and BCR signaling. This includes CD28-mediated activation of PI3K and subsequently Akt, glucose metabolism, and the survival protein Bcl-XL. Overall, this results in the suppression of T-cell or B-cell activation, proliferation, and cytokine secretion. PD-1 expression on T cells following chronic viral infection and on tumor-infiltrating lymphocytes has been shown to result in immune dysfunction characteristic of exhaustion, while blockade of PD-1 signaling has been shown to enhance T-cell proliferation and restore immune responses.

LAG-3 is a transmembrane protein in the Ig superfamily that is expressed on a variety of immune cells. LAG-3 expression occurs both on the cell surface as membrane-bound dimers and in a monomeric soluble form that is the result of cleavage from the membrane by metalloproteinases. LAG-3 is structurally homologous to CD4, containing four extracellular Ig domains. LAG-3 interacts with MHC Class II, but does not compete with CD4 for binding to MHC Class II. Additional LAG-3 binding partners such as liver and lymph node sinusoidal endothelial cell lectin (LSECtin), Galectin-3, and Fibrinogen-like protein 1 (FGL-1) have also been described. Immune suppression by LAG-3 is dependent upon a distinct KIEELE motif in the LAG-3 intracellular domain.

ANTIBODY A is a human Fc-silenced IgGI bispecific antibody that can simultaneously bind to both PD-1 and LAG-3. ANTIBODY A is designed to block immune inhibitory interactions between PD-1 and its two ligands, PD-LI and PD-L2, as well as the inhibitory interactions between LAG-3 and MHC Class II.

The amino acid sequences of the ANTIBODY A heavy and light chains are shown below: ANTIBODY A contains two different heavy chains, a PD-1 heavy chain (which binds to PD-1) and a LAG-3 heavy chain (which binds to LAG-3), and a common light chain that pairs with each of the PD-1 and LAG-3 heavy chains. Complementarity-determining regions (CDRs) 1, 2, and 3 (specified according to Kabat: see Kabat et al., Sequences of Proteins of Immunological Interest, U.S. Dept. of Health and Human Services, Public Health Service. National Institutes of Health, 1991, (OCoLC)1138727707) of the variable heavy (VH) domain and the variable light (VL) domain are shown in that order from N-terminus to the C-terminus of the mature VH and VL sequences and are both underlined and boldened. Variable regions are underlined. An antibody consisting of the PD-I heavy chain amino acid sequence set forth in SEQ ID NO: 1, the LAG-3 heavy chain amino acid sequence set forth in SEQ ID NO:2, and the common light chain amino acid sequence set forth in SEQ ID NO:3 (one light chain paired with each of the heavy chains) is termed “ANTIBODY A.”

ANTIBODY A PD-1 Heavy Chain:
(SEQ ID NO: 1)
QVQLQESGPGLVKPSETLSLTCTVSEGSIGYHFWSWIRQPPGRGLEWIG
YIVYSGSYNVNPSLKTRVTMSVDTSKNQFSLNLRSVTAADTAVYYCARG
GYTGYGGDWFDPWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPELGRGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTDPPSREEMTKNQVSLTCEVKGFYPSDIAVEWESNGQPE
NNYKTTPPVLDSDGSFFLYSKLTVDKSRWQQGNVFSCSVMHEALHNHYT
QKSLSLSPG
ANTIBODY A LAG-3 Heavy Chain:
(SEQ ID NO: 2)
QVQLVQSGSELKKPGASVKVSCKASGYTFTTNALNWVRQAPGQGLEWMG
WINTHTGNPTYAQGFIGRFVFSLDTSVSTAYLQIRSLKAEDTAVYYCAR
EPNWGVYFDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICKVNHKPSNTKVDKRVEPKSCDKTHTCPPCPAPELGRGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTKPPSREEMTKNQVSLKCLVKGFYPSDIAVEWESNGQPENN
YKTTPPVLDSDGSFFLYSKLTVDKSRWQQGNVFSCSVMHEALHNHYTQK
SLSLSPG
ANTIBODY A Common Light Chain:
(SEQ ID NO: 3)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The VH and VL, as well as the CDRs of each of the VH and VL, are depicted in Tables 1 and 2 for the anti-PD-1 binding domain and the anti-LAG-3 binding domain of ANTIBODY A, respectively.

TABLE 1
Amino Acid Sequences of Anti-PD-1 Binding Domain of ANTIBODY A

| Domain | Sequence |
|---|---|
| VH | QVQLQESGPGLVKPSETLSLTCTVSEGSIGYHFWSWIRQPPGRGLEWI |
| | GYIVYSGSYNVNPSLKTRVTMSVDTSKNQFSLNLRSVTAADTAVYYC |
| | ARGGYTGYGGDWFDPWGQGTLVTVSS (SEQ ID NO: 4) |
| VL | DIQMTQSPSSLSASVGDRVTITCRASQSISSYLNWYQQKPGKAPKLLIY |
| | AASSLQSGVPSRFSGSGSGTDFTLTISSLQPEDFATYYCQQSYSTPPTFG |
| | QGTKVEIK (SEQ ID NO: 6) |
| HCDR1 | YHFWS (SEQ ID NO: 7) |
| HCDR2 | YIVYSGSYNVNPSLKT (SEQ ID NO: 8) |
| HCDR3 | GGYTGYGGDWFDP (SEQ ID NO: 9) |
| LCDR1 | RASQSISSYLN (SEQ ID NO: 13) |
| LCDR2 | AASSLQS (SEQ ID NO: 14) |
| LCDR3 | QQSYSTPPT (SEQ ID NO: 15) |

TABLE 2
Amino Acid Sequences of Anti-LAG-3 Binding Domain of ANTIBODY A

| Domain | Sequence |
|---|---|
| VH | QVQLVQSGSELKKPGASVKVSCKASGYTFTTNALNWVRQAPGQGLE |
| | WMGWINTHTGNPTYAQGFIGRFVFSLDTSVSTAYLQIRSLKAEDTAV |
| | YYCAREPNWGVYFDYWGQGTLVTVSS (SEQ ID NO: 5) |
| VL | DIQMTQSPSSLSASVGDRVTITCRASQSISSYLNWYQQKPGKAPKLLIY |
| | AASSLQSGVPSRFSGSGSGTDFTLTISSLQPEDFATYYCQQSYSTPPTFG |
| | QGTKVEIK (SEQ ID NO: 6) |
| HCDR1 | TNALN (SEQ ID NO: 10) |
| HCDR2 | WINTHTGNPTYAQGFIG (SEQ ID NO: 11) |
| HCDR3 | EPNWGVYFDY (SEQ ID NO: 12) |
| LCDR1 | RASQSISSYLN (SEQ ID NO: 13) |
| LCDR2 | AASSLQS (SEQ ID NO: 14) |
| LCDR3 | QQSYSTPPT (SEQ ID NO: 15) |

In certain embodiments, the anti-human PD-1 binding domain of the bispecific antibody of the present disclosure comprises a heavy chain variable region comprising an HCDR1 comprising the amino acid sequence set forth in SEQ ID NO:7, an HCDR2 comprising the amino acid sequence set forth in SEQ ID NO:8, and an HCDR3 comprising the amino acid sequence set forth in SEQ ID NO:9. In certain embodiments, the anti-human PD-1 binding domain of the bispecific antibody of the present disclosure comprises a heavy chain variable region comprising the amino acid sequence as set forth in SEQ ID NO:4, or having at least 80%, 85%, 90%, or 95% sequence identity thereto. In some embodiments, the anti-human PD-1 binding domain of the bispecific antibody of the present disclosure comprises a heavy chain variable region (1) comprising an HCDR1 comprising the amino acid sequence set forth in SEQ ID NO:7, an HCDR2 comprising the amino acid sequence set forth in SEQ ID NO:8, and an HCDR3 comprising the amino acid sequence set forth in SEQ ID NO:9, and (2) comprising the amino acid sequence as set forth in SEQ ID NO:4, or having at least 80%, 85%, 90%, or 95% sequence identity thereto.

In certain embodiments, the anti-human LAG-3 binding domain of the bispecific antibody of the present disclosure comprises a heavy chain variable region comprising an HCDR1 comprising the amino acid sequence set forth in SEQ ID NO: 10, an HCDR2 comprising the amino acid sequence set forth in SEQ ID NO: 11, and an HCDR3 comprising the amino acid sequence set forth in SEQ ID NO:12. In certain embodiments, the anti-human LAG-3 binding domain of the bispecific antibody of the present disclosure comprises a heavy chain variable region comprising the amino acid sequence as set forth in SEQ ID NO:5, or having at least 80%, 85%, 90%, or 95% sequence identity thereto. In some embodiments, the anti-human LAG-3 binding domain of the bispecific antibody of the present disclosure comprises a heavy chain variable region (1) comprising an HCDR1 comprising the amino acid sequence set forth in SEQ ID NO:10, an HCDR2 comprising the amino acid sequence set forth in SEQ ID NO:11, and an HCDR3 comprising the amino acid sequence set forth in SEQ ID NO: 12, and (2) comprising the amino acid sequence as set forth in SEQ ID NO:5, or having at least 80%, 85%, 90%, or 95% sequence identity thereto.

In certain embodiments, the anti-human PD-1 binding domain of the bispecific antibody of the present disclosure comprises a heavy chain variable region comprising an HCDR1 comprising the amino acid sequence set forth in SEQ ID NO: 7, an HCDR2 comprising the amino acid sequence set forth in SEQ ID NO:8, and an HCDR3 comprising the amino acid sequence set forth in SEQ ID NO:9, and the anti-human LAG-3 binding domain of the bispecific antibody comprises a heavy chain variable region comprising an HCDR1 comprising the amino acid sequence set forth in SEQ ID NO: 10, an HCDR2 comprising the amino acid sequence set forth in SEQ ID NO: 11, and an HCDR3 comprising the amino acid sequence set forth in SEQ ID NO: 12. In certain embodiments, the anti-human PD-1 binding domain of the bispecific antibody of the present disclosure comprises a heavy chain variable region comprising the amino acid sequence as set forth in SEQ ID NO:4, or having at least 80%, 85%, 90%, or 95% sequence identity thereto, and the anti-human LAG-3 binding domain of the bispecific antibody comprises a heavy chain variable region comprising the amino acid sequence as set forth in SEQ ID NO:5, or having at least 80%, 85%, 90%, or 95% sequence identity thereto. In some embodiments, the anti-human PD-1 binding domain of the bispecific antibody of the present disclosure comprises a heavy chain variable region (1) comprising an HCDR1 comprising the amino acid sequence set forth in SEQ ID NO:7, an HCDR2 comprising the amino acid sequence set forth in SEQ ID NO:8, and an HCDR3 comprising the amino acid sequence set forth in SEQ ID NO:9, and (2) comprising the amino acid sequence as set forth in SEQ ID NO:4, or having at least 80%, 85%, 90%, or 95% sequence identity thereto, and the anti-human LAG-3 binding domain of the bispecific antibody comprises a heavy chain variable region (1) comprising an HCDRI comprising the amino acid sequence set forth in SEQ ID NO: 10, an HCDR2 comprising the amino acid sequence set forth in SEQ ID NO: 11, and an HCDR3 comprising the amino acid sequence set forth in SEQ ID NO: 12, and (2) comprising the amino acid sequence as set forth in SEQ ID NO:5, or having at least 80%, 85%, 90%, or 95% sequence identity thereto.

In certain embodiments, the anti-human PD-1 binding domain of the bispecific antibody of the present disclosure comprises (1) a heavy chain variable region comprising an HCDR1 comprising the amino acid sequence set forth in SEQ ID NO: 7, an HCDR2 comprising the amino acid sequence set forth in SEQ ID NO:8, and an HCDR3 comprising the amino acid sequence set forth in SEQ ID NO:9, and (2) a light chain variable region comprising an HCDR1 comprising the amino acid sequence set forth in SEQ ID NO: 13, an HCDR2 comprising the amino acid sequence set forth in SEQ ID NO: 14, and an HCDR3 comprising the amino acid sequence set forth in SEQ ID NO: 15. In certain embodiments, the anti-human PD-1 binding domain of the bispecific antibody of the present disclosure comprises (1) a heavy chain variable region comprising the amino acid sequence as set forth in SEQ ID NO:4, or having at least 80%, 85%, 90%. or 95% sequence identity thereto. and (2) a light chain variable region comprising the amino acid sequence as set forth in SEQ ID NO:6. or having at least 80%, 85%, 90%, or 95% sequence identity thereto. In some embodiments, the anti-human PD-1 binding domain of the bispecific antibody of the present disclosure comprises: a heavy chain variable region (1) comprising an HCDR1 comprising the amino acid sequence set forth in SEQ ID NO:7, an HCDR2 comprising the amino acid sequence set forth in SEQ ID NO:8, and an HCDR3 comprising the amino acid sequence set forth in SEQ ID NO:9, and (2) comprising the amino acid sequence as set forth in SEQ ID NO:4. or having at least 80%, 85%, 90%. or 95% sequence identity thereto: and a light chain variable region (1) comprising an HCDR1 comprising the amino acid sequence set forth in SEQ ID NO: 13, an HCDR2 comprising the amino acid sequence set forth in SEQ ID NO: 14. and an HCDR3 comprising the amino acid sequence set forth in SEQ ID NO: 15, and (2) comprising the amino acid sequence as set forth in SEQ ID NO:6, or having at least 80%. 85%, 90%, or 95% sequence identity thereto.

In certain embodiments, the anti-human LAG-3 binding domain of the bispecific antibody of the present disclosure comprises (1) a heavy chain variable region comprising an HCDR1 comprising the amino acid sequence set forth in SEQ ID NO: 10, an HCDR2 comprising the amino acid sequence set forth in SEQ ID NO:11, and an HCDR3 comprising the amino acid sequence set forth in SEQ ID NO:12, and (2) a light chain variable region comprising an HCDR1 comprising the amino acid sequence set forth in SEQ ID NO: 13, an HCDR2 comprising the amino acid sequence set forth in SEQ ID NO: 14, and an HCDR3 comprising the amino acid sequence set forth in SEQ ID NO:15. In certain embodiments. the anti-human LAG-3 binding domain of the bispecific antibody of the present disclosure comprises (1) a heavy chain variable region comprising the amino acid sequence as set forth in SEQ ID NO:5, or having at least 80%, 85%, 90%, or 95% sequence identity thereto, and (2) a light chain variable region comprising the amino acid sequence as set forth in SEQ ID NO:6, or having at least 80%, 85%, 90%, or 95% sequence identity thereto. In some embodiments, the anti-human LAG-3 binding domain of the bispecific antibody of the present disclosure comprises: a heavy chain variable region (1) comprising an HCDRI comprising the amino acid sequence set forth in SEQ ID NO: 10, an HCDR2 comprising the amino acid sequence set forth in SEQ ID NO: 11, and an HCDR3 comprising the amino acid sequence set forth in SEQ ID NO:12, and (2) comprising the amino acid sequence as set forth in SEQ ID NO:5, or having at least 80%, 85%, 90%, or 95% sequence identity thereto: and a light chain variable region (1) comprising an HCDR1 comprising the amino acid sequence set forth in SEQ ID NO: 13, an HCDR2 comprising the amino acid sequence set forth in SEQ ID NO: 14, and an HCDR3 comprising the amino acid sequence set forth in SEQ ID NO: 15, and (2) comprising the amino acid sequence as set forth in SEQ ID NO:6, or having at least 80%. 85%, 90%, or 95% sequence identity thereto.

In certain embodiments, the anti-human PD-1 binding domain of the bispecific antibody of the present disclosure comprises (1) a heavy chain variable region comprising an HCDR1 comprising the amino acid sequence set forth in SEQ ID NO:7, an HCDR2 comprising the amino acid sequence set forth in SEQ ID NO:8, and an HCDR3 comprising the amino acid sequence set forth in SEQ ID NO:9, and (2) a light chain variable region comprising an HCDR1 comprising the amino acid sequence set forth in SEQ ID NO: 13, an HCDR2 comprising the amino acid sequence set forth in SEQ ID NO: 14, and an HCDR3 comprising the amino acid sequence set forth in SEQ ID NO: 15, and the anti-human LAG-3 binding domain of the bispecific antibody comprises (1) a heavy chain variable region comprising an HCDR1 comprising the amino acid sequence set forth in SEQ ID NO:10, an HCDR2 comprising the amino acid sequence set forth in SEQ ID NO: 11, and an HCDR3 comprising the amino acid sequence set forth in SEQ ID NO: 12, and (2) a light chain variable region comprising an HCDR1 comprising the amino acid sequence set forth in SEQ ID NO: 13, an HCDR2 comprising the amino acid sequence set forth in SEQ ID NO: 14, and an HCDR3 comprising the amino acid sequence set forth in SEQ ID NO: 15. In certain embodiments, the anti-human PD-1 binding domain of the bispecific antibody of the present disclosure comprises (1) a heavy chain variable region comprising the amino acid sequence as set forth in SEQ ID NO:4, or having at least 80%, 85%, 90%, or 95% sequence identity thereto, and (2) a light chain variable region comprising the amino acid sequence as set forth in SEQ ID NO:6, or having at least 80%, 85%, 90%, or 95% sequence identity thereto, and the anti-human LAG-3 binding domain of the bispecific antibody comprises (1) a heavy chain variable region comprising the amino acid sequence as set forth in SEQ ID NO:5, or having at least 80%, 85%, 90%, or 95% sequence identity thereto, and (2) a light chain variable region comprising the amino acid sequence as set forth in SEQ ID NO:6, or having at least 80%, 85%, 90%, or 95% sequence identity thereto. In some embodiments, the anti-human PD-1 binding domain of the bispecific antibody of the present disclosure comprises: a heavy chain variable region (1) comprising an HCDR1 comprising the amino acid sequence set forth in SEQ ID NO:7, an HCDR2 comprising the amino acid sequence set forth in SEQ ID NO:8, and an HCDR3 comprising the amino acid sequence set forth in SEQ ID NO:9, and (2) comprising the amino acid sequence as set forth in SEQ ID NO:4, or having at least 80%. 85%, 90%, or 95% sequence identity thereto: and a light chain variable region (1) comprising an HCDR1 comprising the amino acid sequence set forth in SEQ ID NO: 13, an HCDR2 comprising the amino acid sequence set forth in SEQ ID NO: 14, and an HCDR3 comprising the amino acid sequence set forth in SEQ ID NO:15, and (2) comprising the amino acid sequence as set forth in SEQ ID NO:6, or having at least 80%, 85%, 90%, or 95% sequence identity thereto, and the anti-human LAG-3 binding domain of the bispecific antibody comprises: a heavy chain variable region (1) comprising an HCDR1 comprising the amino acid sequence set forth in SEQ ID NO: 10, an HCDR2 comprising the amino acid sequence set forth in SEQ ID NO:11, and an HCDR3 comprising the amino acid sequence set forth in SEQ ID NO: 12, and (2) comprising the amino acid sequence as set forth in SEQ ID NO:5, or having at least 80%, 85%, 90%, or 95% sequence identity thereto: and a light chain variable region (1) comprising an HCDR1 comprising the amino acid sequence set forth in SEQ ID NO: 13, an HCDR2 comprising the amino acid sequence set forth in SEQ ID NO: 14, and an HCDR3 comprising the amino acid sequence set forth in SEQ ID NO: 15, and (2) comprising the amino acid sequence as set forth in SEQ ID NO:6, or having at least 80%, 85%, 90%, or 95% sequence identity thereto.

In certain embodiments, the bispecific antibody includes a human heavy chain and light chain constant region. In certain embodiments, the heavy chain constant region comprises a CHI domain and a hinge region. In some embodiments, the heavy chain constant region comprises a CH2 domain. In some embodiments, the heavy chain constant region comprises a CH3 domain. In some embodiments, the heavy chain constant region comprises CHI, CH2 and CH3 domains. If the heavy chain constant region includes substitutions, such substitutions modify the properties of the antibody (e.g., increase or decrease one or more of: Fc receptor binding, antibody glycosylation, the number of cysteine residues, effector cell function, or complement function). In certain embodiments, the antibody is an IgG antibody. In specific embodiments, the antibody is selected from the group consisting of IgGI, IgG2, IgG3, and IgG4.

Antibodies, such as ANTIBODY A, can be made, for example, by preparing and expressing synthetic genes that encode the recited amino acid sequences or by mutating human germline genes to provide a gene that encodes the recited amino acid sequences. Moreover, this antibody and other bispecific antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science. 229:1202-1207 (1985), by Oi et al .. BioTechniques. 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992): Cook, G. P. et al., Immunol. Today. 16: 237-242 (1995): Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861: U.S. Pat. Nos. 5,693,762: 5,693,761: 5,585,089; 5,530,101: and 6,407,213: Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

Provided herein are compositions comprising a mixture of a bispecific antibody and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising a bispecific antibody comprising at least one deamidation site, wherein the pH of the composition is from about 5.0 to about 6.5, such that, e.g., at least about 90% of the bispecific antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising a bispecific antibody means the presence of both the desired bispecific antibody and one or more acidic variants thereof. The acidic variants may comprise predominantly deamidated bispecific antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

Bispecific antibodies of the disclosure can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Methods of Producing Antibodies

Antibodies may be produced in, for example, bacterial or eukaryotic cells. Some antibodies can be produced in bacterial cells, e.g., E. coli cells. Antibodies can also be produced in eukaryotic cells such as transformed cell lines (e.g., CHO, 293E, COS). In addition, antibodies can be expressed in a yeast cell such as Pichia (see, e.g., Powers et al., J Immunol Methods. 251: 123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell

If the antibody is to be expressed in animal cells such as CHO, COS, and NIH3T3 cells, the expression vector includes a promoter necessary for expression in these cells, for example, an SV40 promoter (Mulligan et al., Nature, 277: 108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, POPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector(s) encoding the antibody heavy chains and the antibody light chains of a bispecific antibody (e.g., ANTIBODY A) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHER gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography; ion exchange chromatography, hydrophobic chromatography; gel filtration, reverse-phase chromatography; and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

A bispecific antibody described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472): Ansel et al., Pharmaceutical Dosage Forms and Drug Delivery Systems, 7^{th} Ed., Lippincott Williams & Wilkins Publishers (1999) (ISBN: 0683305727): and Kibbe (ed.), Handbook of Pharmaceutical Excipients American Pharmaceutical Association, 3^{rd} ed. (2000) (ISBN: 091733096X).

The bispecific antibody can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody can also be tailored for the individual case, e.g., by monitoring the subject, e.g., using tomographic imaging, e.g., to visualize a tumor.

The bispecific antibody can be administered as a fixed dose, or in a mg/kg patient weight dose. The dose can also be chosen to reduce or avoid production of antibodies against the bispecific antibody. Dosage regimens are adjusted to provide the desired response, e.g., a therapeutic response or a combinatorial therapeutic effect. Generally, doses of the bispecific antibody can be used in order to provide a subject with the agent in bioavailable quantities.

For example, doses in the range of about 0.1 mg/kg to about 30 mg/kg can be administered. In specific embodiments, a subject is administered the antibody at a dose of about 0.1 mg/kg to about 10 mg/kg (e.g., a dose of about 0.1 mg/kg, 0.5 mg/kg, 1 mg/kg, 1.5 mg/kg, 2 mg/kg, 3 mg/kg, 5 mg/kg, 6 mg/kg, 7.5 mg/kg, or about 10 mg/kg). In other embodiments, a subject is administered the antibody at a dose of about 1 mg/kg to about 3 mg/kg (e.g., a dose of about 1 mg/kg, 2 mg/kg, or 3 mg/kg). With respect to doses or dosages, the term “about” is intended to denote a range that is ±10% of a recited dose, such that, for example, a dose of about 3 mg/kg will be between 2.7 mg/kg and 3.3 mg/kg patient weight.

Dosage unit form or “fixed dose” or “flat dose” as used herein refers to physically discrete units suited as unitary dosages for the subjects to be treated: each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion. For example, flat doses in the range of about 20 mg to 2500 mg can be administered. In specific embodiments, a subject is administered the antibody at a dose of about 20 mg, 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, or 2500 mg. In other embodiments, a subject is administered the antibody at a dose of about 20 mg, 50 mg, 150 mg, 450 mg, 900 mg, 1800 mg, or 2500 mg.

A bispecific antibody dose can be administered, e.g., at a periodic interval over a period of time (a course of treatment) sufficient to encompass at least 2 doses, 3 doses, 5 doses, 10 doses, or more, e.g., weekly, biweekly (every two weeks), every three weeks, every four weeks, monthly, e.g., for between about 1 to 12 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

An exemplary flat dose dosing regimen comprises intravenous administration of a bispecific antibody described herein (e.g., ANTIBODY A) at a dosage of about 20 mg once every three weeks.

A further exemplary flat dose dosing regimen comprises intravenous administration of a bispecific antibody described herein (e.g., ANTIBODY A) at a dosage of about 50 mg once every three weeks.

A further exemplary flat dose dosing regimen comprises intravenous administration of a bispecific antibody described herein (e.g., ANTIBODY A) at a dosage of about 150 mg once every three weeks.

A further exemplary flat dose dosing regimen comprises intravenous administration of a bispecific antibody described herein (e.g., ANTIBODY A) at a dosage of about 450 mg once every three weeks.

A further exemplary flat dose dosing regimen comprises intravenous administration of a bispecific antibody described herein (e.g., ANTIBODY A) at a dosage of about 900 mg once every three weeks.

A further exemplary flat dose dosing regimen comprises intravenous administration of a bispecific antibody described herein (e.g., ANTIBODY A) at a dosage of about 1800 mg once every three weeks.

A further exemplary flat dose dosing regimen comprises intravenous administration of a bispecific antibody described herein (e.g., ANTIBODY A) at a dosage of about 2500 mg once every three weeks.

A pharmaceutical composition may include a “therapeutically effective amount” of a bispecific antibody described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Indications

A bispecific antibody described herein (e.g., ANTIBODY A) can be used to treat melanoma. In some embodiments, the melanoma is unresectable or metastatic melanoma. In some embodiments, the melanoma has a V600-activating BRAF mutation. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat squamous cell carcinoma of the head and neck (SCCHN). In some embodiments, the SCCHN is recurrent or metastatic SCCHN. In some embodiments, the SCCHN is PD-L1+ SCCHN (combined positive score ≥1). In some embodiments, the SCCHN is a primary squamous tumor of the oral cavity, oropharynx, hypopharynx, or larynx. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat NSCLC. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat small cell lung cancer. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat malignant pleural mesothelioma. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat hormone receptor-positive/HER2 negative breast cancer. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat triple-negative breast cancer. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat nasopharyngeal carcinoma. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat esophageal carcinoma. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat hepatocellular carcinoma. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat renal cell carcinoma. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat urothelial carcinoma. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat a B-cell lymphoma. In some embodiments, the B-cell lymphoma is DLBCL or PMBCL. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat a MSI-H/dMMR solid tumor. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat a SCCHN, anal carcinoma, or cervical cancer. In some embodiments, the SCCHN, anal carcinoma, or cervical cancer is HPV-positive SCCHN, anal carcinoma, or cervical cancer. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat a DNA polymerase epsilon mutated solid tumor. In some embodiments, the DNA polymerase epsilon mutated solid tumor comprises DNA polymerase epsilon mutations P286R and/or V411L. In some embodiments, the subject has experienced disease progression after prior treatment (e.g., prior anti-PD-(L)1 therapy and/or and platinum-based therapy).

A bispecific antibody described herein (e.g., ANTIBODY A) can be used to treat clear cell ovarian or endometrial carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of melanoma. In some embodiments, the melanoma is unresectable or metastatic melanoma. In some embodiments, the melanoma has a V600-activating BRAF mutation. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of SCCHN. In some embodiments, the SCCHN is recurrent or metastatic SCCHN. In some embodiments, the SCCHN is PD-L1+ SCCHN (combined positive score ≥1). In some embodiments, the SCCHN is a primary squamous tumor of the oral cavity, oropharynx, hypopharynx, or larynx. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of NSCLC. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of small cell lung cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of malignant pleural mesothelioma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of hormone receptor-positive/HER2 negative breast cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of triple-negative breast cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of nasopharyngeal carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of esophageal carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of hepatocellular carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of renal cell carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of urothelial carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of a B-cell lymphoma. In some embodiments, the B-cell lymphoma is DLBCL or PMBCL. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of a MSI-H/dMMR solid tumor. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of a SCCHN, anal carcinoma, or cervical cancer. In some embodiments, the SCCHN, anal carcinoma, or cervical cancer is HPV-positive SCCHN, anal carcinoma, or cervical cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of a DNA polymerase epsilon mutated solid tumor. In some embodiments, the DNA polymerase epsilon mutated solid tumor comprises DNA polymerase epsilon mutations P286R and/or V411L. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) for use in the treatment of clear cell ovarian or endometrial carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating melanoma. In some embodiments, the melanoma is unresectable or metastatic melanoma. In some embodiments, the melanoma has a V600-activating BRAF mutation. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating SCCHN. In some embodiments, the SCCHN is recurrent or metastatic SCCHN. In some embodiments, the SCCHN is PD-L1+ SCCHN (combined positive score ≥1). In some embodiments, the SCCHN is a primary squamous tumor of the oral cavity, oropharynx, hypopharynx, or larynx. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating NSCLC. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating small cell lung cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating malignant pleural mesothelioma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating hormone receptor-positive/HER2 negative breast cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating triple-negative breast cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating nasopharyngeal carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating esophageal carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating hepatocellular carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating renal cell carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating urothelial carcinoma. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating a B-cell lymphoma. In some embodiments, the B-cell lymphoma is DLBCL or PMBCL. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating a MSI-H/dMMR solid tumor. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating a SCCHN, anal carcinoma, or cervical cancer. In some embodiments, the SCCHN, anal carcinoma, or cervical cancer is HPV-positive SCCHN, anal carcinoma, or cervical cancer. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating a DNA polymerase epsilon mutated solid tumor. In some embodiments, the DNA polymerase epsilon mutated solid tumor comprises DNA polymerase epsilon mutations P286R and/or V411L. In some embodiments, the subject has experienced disease progression after prior treatment (e.g., prior anti-PD-(L)1 therapy and/or and platinum-based therapy).

Another aspect comprises a bispecific antibody described herein (e.g., ANTIBODY A) in the manufacture of a medicament for treating clear cell ovarian or endometrial carcinoma. In some embodiments, the subject has experienced disease progression after prior treatment (e.g., prior anti-PD-(L)1 therapy and/or and platinum-based therapy).

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: A Study of ANTIBODY A in Participants With Select Advanced Malignancies

This is a phase 1, multicenter, open-label, dose-escalation, and dose-expansion clinical study to investigate the safety, tolerability, pharmacokinetic (PK), pharmacodynamics, and preliminary clinical efficacy of ANTIBODY A in participants with selected advanced malignancies.

Part 1 of the study is a dose escalation in participants with select advanced malignancies. Part I will assess the safety and tolerability and identify the maximum tolerated dose (MTD) and/or recommended dose for expansion (RDE). The select advanced malignancies included in Part 1 are melanoma, non-small cell lung cancer (NSCLC), small cell lung cancer, malignant pleural mesothelioma, hormone receptor-positive/human epidermal growth factor receptor 2 (HER2) negative breast cancer, triple-negative breast cancer, nasopharyngeal carcinoma, esophageal carcinoma, hepatocellular carcinoma, renal cell carcinoma, urothelial carcinoma, select B-cell lymphomas (including diffuse large B-cell lymphoma (DLBCL) and primary mediastinal B-cell lymphoma (PMBCL)), high microsatellite instability (MSI-H)/deficient mismatch repair (dMMR) solid tumors, human papilloma virus (HPV)-positive squamous cell carcinoma of the head and neck (SCCHN), anal carcinoma, or cervical cancer, DNA polymerase epsilon mutated solid tumors (P286R and V411L), clear cell ovarian or endometrial carcinoma, and other tumor types with medical monitor approval.

Part 2 of the study is an open-label dose expansion to further evaluate the safety, tolerability, PK, pharmacodynamics, and preliminary antitumor activity of ANTIBODY A at selected RDE(s) in two tumor-specific cohorts:
Cohort 1: Participants with histologically or cytologically confirmed Stage III (unresectable) or Stage IV (metastatic) melanoma that is considered nonamenable to curative treatments or procedures and who have experienced disease progression after treatment with standard therapy. Participants must have documentation of V600-activating BRAF mutation status or consent to BRAF V600 mutation testing during the screening period.
Cohort 2: Participants with histologically or cytologically confirmed recurrent/metastatic (R/M) SCCHN that is PD-L1 positive (CPS ≥1) (including primary tumors of the oral cavity, oropharynx, hypopharynx, or larynx) that is not amenable to local therapy with curative intent (surgery or radiation with or without chemotherapy) and who have experienced disease progression after treatment with standard therapy.

For Part 2, prior treatment with “standard therapy” includes available standard therapies, including anti-PD-(L)1 and platinum-based therapy, that are known to confer clinical benefit.

### Justification for Dose

For Part 1, an ANTIBODY A starting dose of 20 mg once every 3 weeks (Q3W) was selected since this dose is predicted to result in an average concentration that is consistent with the EC50 for enhanced staphylococcal enterotoxin B (SEB)-stimulated IL-2 secretion from human peripheral blood mononuclear cells (PBMCs) and reduction of free/total PD-1 in vivo.

The proposed safe starting dose (SSD) attempts to minimize the exposure of patients with advanced cancer to subtherapeutic dose levels of ANTIBODY A while balancing the safety risk associated with the nonclinical pharmacologic and toxicological profiles. Based on this assessment, the SSD is expected to be safe for the following reasons:
(1) The SSD (20 mg Q3W) is about 96-fold lower than the human equivalent dose at the highest dose tested in the 4-week Good Laboratory Practice (GLP) study (100 mg/kg once weekly: human equivalent dose: 1920 mg). The only adverse finding at this dose was moderate renal tubular necrosis in a single animal that was considered likely the result of a thrombotic/embolic event secondary to vascular inflammation associated with antidrug antibody (ADA) formation and immune complex deposition and thus of uncertain relevance to human participants. In addition, the SSD (20 mg Q3W) is about 3-fold lower than the no-observed-adverse-effect level in the 4-week GLP study (3 mg/kg twice weekly: human equivalent dose: 58 mg). The SSD is below the maximum SSD of 300 mg/dose allowed under the ICH S9 guideline (ICH 2009) for determining the SSD for oncology agents.

Moreover, ANTIBODY A was administered once weekly to cynomolgus monkeys and will initially be administered Q3W to study participants, providing an additional margin of safety.
(2) There is an absence of evidence for cytokine release that would be predictive of cytokine release syndrome in an in vitro whole blood cytokine release assay and no significant cytokine release in cynomolgus monkeys following administration of ANTIBODY A.
(3) There was no unexpected tissue staining in human tissues in a tissue cross-reactivity study.
(4) The SSD of 20 mg Q3W is projected to produce serum concentrations that provide 180 hours (out of 504 hours) coverage of the ECso for enhanced SEB-stimulated IL-2 secretion from human PBMCs while not reaching EC98 from the SEB-stimulated human PBMC assay and thus is expected to produce suboptimal pharmacology

In Part 2, ANTIBODY A will be administered at the RDE(s) identified in Part 1. In the event that two RDEs are selected for evaluation within a particular dose-expansion cohort, participants will be randomized to receive one of the RDEs during study participation.

### ANTIBODY A Regimen

The starting dose of ANTIBODY A in Part 1 is 20 mg administered by intravenous infusion Q3W. The following additional dose levels will be evaluated during Part 1 of the study: 50 mg, 150 mg, 450 mg, 900 mg, 1800 mg, and 2500 mg.

### Objectives and Endpoints

The primary objective of the study is to evaluate the safety and tolerability and determine the MTD and/or RDE(s) of ANTIBODY A in participants with selected advanced malignancies. The primary objective is evaluated by measuring (1) occurrence of dose-limiting toxicities (DLTs), (2) incidence of treatment-emergent adverse events (TEAEs), assessed by physical examinations, evaluating changes in vital signs and electrocardiograms (ECGs), and clinical laboratory blood sample evaluations, and (3) incidence of TEAEs leading to study drug treatment interruptions and withdrawal of study drug due to adverse events (AEs).

The secondary objectives of the study are: (1) to determine the preliminary efficacy of ANTIBODY A in terms of objective response rate (ORR), disease control rate (DCR), and duration of response (DOR) in participants with selected advanced malignancies, (2) to evaluate the PK of ANTIBODY A in participants with selected advanced malignancies, and (3) to evaluate the target engagement of ANTIBODY A via receptor occupancy in participants with selected advanced malignancies. The secondary objectives are evaluated by measuring the following endpoints: (1) Objective response: complete response (CR) or partial response (PR), as determined by the investigator by radiographic disease assessment according to RECIST v1.1 or Lugano criteria (B-cell lymphomas only), Disease control: CR, PR, or stable disease (SD) as determined by the investigator by radiographic disease assessment according to RECIST v1.1 or Lugano criteria (B-cell lymphomas only), and DOR: time from earliest date of disease response (CR or PR) until earliest date of disease progression as determined by the investigator by radiographic disease assessment according to RECIST v1.1 or Lugano criteria (B-cell lymphomas only), (2) PK parameters for ANTIBODY A, including Cₘₐₓ, tₘₐₓ, Cₘᵢₙ, AUC, CL, V_{z}, and t_{1/2}, as deemed appropriate, and (3) PD-1 receptor occupancy in peripheral blood samples.

The exploratory objectives of the study are: (1) to assess changes in immune profiles and biomarkers of treatment effect from pretreatment and post-treatment blood and tumor tissue in participants and associations with clinical activity, (2) to assess the immunogenicity of ANTIBODY A in participants with selected advanced malignancies, (3) to examine the association of the PK of ANTIBODY A with receptor occupancy in participants with selected advanced malignancies, (4) to explore the relationship between ANTIBODY A exposure and response in participants with selected advanced malignancies, and (5) to explore the relationship between ANTIBODY A exposure and safety in participants with selected advanced malignancies. The exploratory objectives are evaluated by measuring the following endpoints: (1) changes in biomarkers, including but not limited to peripheral immune phenotypes, inflammatory cytokines from baseline to each visit where the variable is measured, and correlation with treatment outcomes, (2) immunogenicity, defined as the occurrence of specific ADAs to ANTIBODY A, (3) the plasma levels of ANTIBODY A will be examined for correlation with a measure of PD-1 receptor occupancy among participants with advanced malignancies, (4) PK and exposure data will be assessed and correlated to tumor measurements, and (5) PK and exposure data will be assessed and correlated to incidence and severity of TEAEs and DLTs.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a disorder in a human subject in need thereof, wherein the disorder is selected from the group consisting of a melanoma, non-small cell lung cancer (NSCLC), small cell lung cancer, malignant pleural mesothelioma, hormone receptor-positive/human epidermal growth factor receptor 2 (HER2) negative breast cancer, triple-negative breast cancer, nasopharyngeal carcinoma, esophageal carcinoma, hepatocellular carcinoma, renal cell carcinoma, urothelial carcinoma, a B-cell lymphoma, a high microsatellite instability (MSI-H)/deficient mismatch repair (dMMR) solid tumor, a squamous cell carcinoma of the head and neck (SCCHN), anal carcinoma, cervical cancer, a DNA polymerase epsilon mutated solid tumor, and clear cell ovarian or endometrial carcinoma, wherein the method comprises administering to the human subject a therapeutically effective amount of a bispecific antibody that binds to human PD-1 and human LAG-3, wherein the bispecific antibody comprises:
an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence YHFWS (SEQ ID NO:7), heavy chain CDR2 (HCDR2) comprising the amino acid sequence YIVYSGSYNVNPSLKT (SEQ ID NO:8), and heavy chain CDR3 (HCDR3) comprising the amino acid sequence GGYTGYGGDWFDP (SEQ ID NO:9), and wherein the PD-1 light chain variable region comprises light chain CDRI (LCDRI) comprising the amino acid sequence RASQSISSYLN (SEQ ID NO: 13), light chain CDR2 (LCDR2) comprising the amino acid sequence AASSLQS (SEQ ID NO:14), and light chain CDR3 (LCDR3) comprising the amino acid sequence QQSYSTPPT (SEQ ID NO:15): and
an anti-human LAG-3 binding domain comprising a LAG-3 heavy chain variable region and a LAG-3 light chain variable region, wherein the LAG-3 heavy chain variable region comprises HCDR1 comprising the amino acid sequence TNALN (SEQ ID NO:10), HCDR2 comprising the amino acid sequence WINTHTGNPTYAQGFIG (SEQ ID NO: 11), and HCDR3 comprising the amino acid sequence EPNWGVYFDY (SEQ ID NO:12), and wherein the LAG-3 light chain variable region comprises LCDRI comprising the amino acid sequence RASQSISSYLN (SEQ ID NO: 13), LCDR2 comprising the amino acid sequence AASSLQS (SEQ ID NO: 14), and LCDR3 comprising the amino acid sequence QQSYSTPPT (SEQ ID NO:15).

2. The method of claim 1, wherein the PD-1 heavy chain variable region comprises the amino acid sequence
(SEQ ID NO: 4)
QVQLQESGPGLVKPSETLSLTCTVSEGSIGYHFWSWIRQPPGRGLEWIG
YIVYSGSYNVNPSLKTRVTMSVDTSKNQFSLNLRSVTAADTAVYYCARG
GYTGYGGDWFDPWGQGTLVTVSS
and the LAG-3 heavy chain variable region comprises the amino acid sequence
(SEQ ID NO: 5)
QVQLVQSGSELKKPGASVKVSCKASGYTFTTNALNWVRQAPGQGLEWMG
WINTHTGNPTYAQGFIGRFVFSLDTSVSTAYLQIRSLKAEDTAVYYCAR
EPNWGVYFDYWGQGTLVTVSS

3. The method of claim 1 or 2, wherein the PD-1 light chain variable region and the LAG-3 light chain variable region each comprise the amino acid sequence
(SEQ ID NO: 6)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK

4. The method of claim 1, wherein the bispecific antibody comprises a PD-1 heavy chain, a PD-1 light chain, a LAG-3 heavy chain, and a LAG-3 light chain, wherein the PD-1 heavy chain comprises the amino acid sequence set forth in SEQ ID NO:1, the PD-1 light chain light chain comprises the amino acid sequence set forth in SEQ ID NO:3, the LAG-3 heavy chain comprises the amino acid sequence set forth in SEQ ID NO:2, and the LAG-3 light chain light chain comprises the amino acid sequence set forth in SEQ ID NO:3.

5. The method of any one of claims 1 to 4, wherein the human subject has a melanoma.

6. The method of claim 5, wherein the melanoma is unresectable or metastatic melanoma.

7. The method of claim 5 or 6, wherein the melanoma has a V600-activating BRAF mutation.

8. The method of any one of claims 1 to 4, wherein the human subject has a SCCHN.

9. The method of claim 8, wherein the SCCHN is recurrent or metastatic SCCHN.

10. The method of claim 8 or 9, wherein the SCCHN is PD-L1+ SCCHN.

11. The method of any one of claims 8-10, wherein the SCCHN is a primary squamous tumor of the oral cavity, oropharynx, hypopharynx, or larynx.

12. The method of any one of claims 1 to 4, wherein the human subject has NSCLC.

13. The method of any one of claims 1 to 4, wherein the human subject has small cell lung cancer.

14. The method of any one of claims 1 to 4, wherein the human subject has malignant pleural mesothelioma.

15. The method of any one of claims 1 to 4, wherein the human subject has hormone receptor-positive/HER2 negative breast cancer.

16. The method of any one of claims 1 to 4, wherein the human subject has triple-negative breast cancer.

17. The method of any one of claims 1 to 4, wherein the human subject has nasopharyngeal carcinoma.

18. The method of any one of claims 1 to 4, wherein the human subject has esophageal carcinoma.

19. The method of any one of claims 1 to 4, wherein the human subject has hepatocellular carcinoma.

20. The method of any one of claims 1 to 4, wherein the human subject has renal cell carcinoma.

21. The method of any one of claims 1 to 4, wherein the human subject has urothelial carcinoma.

22. The method of any one of claims 1 to 4, wherein the human subject has a B-cell lymphoma.

23. The method claim of 22, wherein the B-cell lymphoma is diffuse large B-cell lymphoma (DLBCL) or primary mediastinal B-cell lymphoma (PMBCL).

24. The method of any one of claims 1 to 4, wherein the human subject has a MSI-H/dMMR solid tumor.

25. The method of any one of claims 1 to 4, wherein the human subject has a SCCHN, anal carcinoma, or cervical cancer.

26. The method of claim 25, wherein the human subject has human papilloma virus (HPV)-positive SCCHN, anal carcinoma, or cervical cancer.

27. The method of any one of claims 1 to 4, wherein the human subject has a DNA polymerase epsilon mutated solid tumor.

28. The method of claim 27, wherein the DNA polymerase epsilon mutated solid tumor comprises DNA polymerase epsilon mutations P286R and/or V411L.

29. The method of any one of claims 1 to 4, wherein the human subject has clear cell ovarian or endometrial carcinoma.

30. The method of any one of the preceding claims, wherein the human subject has experienced disease progression after prior treatment.

31. The method of claim 30, wherein the prior treatment comprises anti-PD-(L)1 therapy and/or and platinum-based therapy.

32. The method of any one of the preceding claims, wherein the disorder is nonamenable to curative treatments or procedures.

33. The method of any one of the preceding claims, wherein the bispecific antibody is administered intravenously.

34. The method of any one of the preceding claims, wherein the bispecific antibody is administered at a dose of about 20 mg, about 50 mg, about 150 mg, about 450 mg, about 900 mg, about 1800 mg, or about 2500 mg.

35. The method of any one of the preceding claims, wherein the bispecific antibody is administered once every three weeks.

36. The method of any one of the preceding claims, wherein the bispecific antibody is administered intravenously at a dose of about 20 mg, about 50 mg, about 150 mg, about 450 mg, about 900 mg, about 1800 mg, or about 2500 mg.

37. The method of any one of the preceding claims, wherein the bispecific antibody is administered intravenously once every three weeks at a dose of about 20 mg, about 50 mg, about 150 mg, about 450 mg, about 900 mg, about 1800 mg, or about 2500 mg.

38. The method of any one of the preceding claims, wherein the bispecific antibody comprises a PD-1 heavy chain, a PD-1 light chain, a LAG-3 heavy chain, and a LAG-3 light chain, wherein the PD-1 heavy chain comprises the amino acid sequence set forth in SEQ ID NO:1, the PD-1 light chain light chain comprises the amino acid sequence set forth in SEQ ID NO:3, the LAG-3 heavy chain comprises the amino acid sequence set forth in SEQ ID NO:2, and the LAG-3 light chain light chain comprises the amino acid sequence set forth in SEQ ID NO:3, and wherein the bispecific antibody is administered intravenously at a dose of about 20 mg, about 50 mg, about 150 mg, about 450 mg, about 900 mg, about 1800 mg, or about 2500 mg.

39. The method of any one of the preceding claims, wherein the bispecific antibody comprises a PD-1 heavy chain, a PD-1 light chain, a LAG-3 heavy chain, and a LAG-3 light chain, wherein the PD-1 heavy chain comprises the amino acid sequence set forth in SEQ ID NO:1, the PD-1 light chain light chain comprises the amino acid sequence set forth in SEQ ID NO:3, the LAG-3 heavy chain comprises the amino acid sequence set forth in SEQ ID NO:2, and the LAG-3 light chain light chain comprises the amino acid sequence set forth in SEQ ID NO:3, and wherein the bispecific antibody is administered intravenously once every three weeks at a dose of about 20 mg, about 50 mg, about 150 mg, about 450 mg, about 900 mg, about 1800 mg, or about 2500 mg.
